(19) United States
(12) Patent Application Publication
(10) Pub. No.: US 2009/0226241 A1
(43) Pub. Date: Sep. 10, 2009

(54) Ultrasonic Toothbrush With Irrigation and Vacuum

(76) Inventors: Mendal Keith McEwen, Westport (CA); William Maynard, Westport (CA)

Correspondence Address: ADAMS PATENT & TRADEMARK AGENCY, P.O. BOX 11100, STATION H, OTTAWA, ON, K2H 7T8, CA

(21) Appl. No.: 12/086,912

(22) PCT Filed: Dec. 21, 2006
(86) PCT No.: PCT/CA2006/002064

§ 371 Date: Jun. 20, 2008

Related U.S. Application Data
(60) Provisional application No. 60/752,588, filed on Dec. 22, 2005.

(51) Int. Cl. A46B 11/00

(52) U.S. Cl. 401/268

(57) ABSTRACT

A toothbrush apparatus comprises a toothbrush head having a bristle portion at its one end portion comprising bristles and at its other end portion a handle. Fluid supply means provides pressurized fluid to the bristle portion and has an inlet connectable to a source of fluid, for example a faucet, and an outlet formed in the bristle portion. Suction means having an inlet associated with the bristle portion evacuates waste from the oral cavity of a user for disposal. An ultrasonic transducer imparts ultrasonic vibrations to the bristle portion and power supply means supplies electrical power to the ultrasonic transducer.

### TECHNICAL FIELD

The invention relates to powered toothbrushes and is especially applicable to toothbrushes that employ ultrasonic vibration for removing plaque, tartar and other debris.

### BACKGROUND ART

It is generally recognized that regular brushing aids in keeping teeth healthy and prevents gum disease. The practice of oral hygiene in the privacy of one's home has been limited to the use of manual and battery-operated toothbrushes, and manual and battery operated floss and flossing devices, and a variety of chemical rinses.

A disadvantage of brushing, however, is that it does not effectively remove hidden debris such as plaque and tartar from between the teeth. As a result, many dental practitioners recommend that brushing be followed up with flossing to loosen and remove such debris. However, flossing removes only a fraction of the loosened material and can cause injury to the soft tissues thereby allowing the entrance of harmful bacteria from the mouth or fingers into the body. Furthermore, rather than removing plaque, flossing and brushing can simply push it around in the oral cavity. This is particularly problematic for persons with gum (periodontal) disease as the plaque can be irretrievably forced into depressions along the gum line (periodontal pockets) which are formed as a result of a chronic disease process that develops by bacteria that feed on retained plaque.

Another disadvantage of brushing is that the bristles of toothbrushes can harbor a number of infectious pathogens and microbes which can be carried into the bloodstream through lesions in the oral cavity. For instance, toothbrushes are reservoirs of pathogens which have been linked to ulcers, digestive tract illnesses, respiratory infections, kidney ailments and, in some cases, serious heart ailments. It has also been reported that vigorous brushing and flossing can cause infective endocarditis, which is a disease caused by infection of the innermost lining of the heart. Patients with heart (valvular) defects who have had rheumatic fever, and those who have had artificial joint replacements within a two year period, are routinely administered a prophylatic dose of antibiotics prior to a cleaning by a dentist or hygienist.

Other methods for cleaning teeth which avoid some of the disadvantages of brushing include systems which make use of a stream of pressurized water which is directed at the surface of the teeth, e.g., the Water Pik™ sold by Teledyne™. The stream of water is created by connecting the system to a source of water under pressure. For more effective cleaning, the pressurized water can be applied intermittently as a pulsating stream. Although such oral irrigation devices are effective in removing relatively loose and bulky plaque and debris from tooth surfaces, they suffer the disadvantage that that they do not necessarily remove plaque that has been retained on the teeth for more than 48 hours. Furthermore, the stream of pressurized water can exacerbate gum disease by pushing loosened plaque irretrievably into any depressions formed along the gum line (periodontal pockets).

It is known to combine irrigation with brushing and power vacuuming. Such vacuuming/irrigation systems have been used in institutions, such as hospitals and long-term facilities, for cleaning the teeth of patients who are unable to care for themselves. Such apparatus required a utility cart to transport the equipment from patient to patient and incorporates a brush, which can inject water under pressure, suction off the dislodged matter and injected liquid and then evacuate it from the mouth. However, although the systems described are effective in an institutional setting, they would not be amenable to home use due to their size. Furthermore, the systems are subject to many of the disadvantages described above in relation to manual toothbrushes.

Yet another method of removing plaque and other debris from teeth is by using ultrasonic energy. Ultrasonic cleaning may be accomplished through the use of the cavitation effect, which is the rapid formation and collapse of tiny gas and vapor filled bubbles or cavities in a solution that is irradiated with ultrasonic energy. The repeated collapsing of these bubbles produces tiny shock waves that remove any contaminants from the surfaces of the teeth and which are effective at penetrating crevices. Examples of such devices are described in U.S. Pat. Nos. 5,448,792, and 5,546,624 which disclose ultrasonic toothbrushes with bristles that resonate at an ultrasonic frequency, and U.S. Pat. No. 6,203,320 which discloses an electric toothbrush with hollow bristles to allow for the passage of irrigating liquid which is pulsated at an ultrasonic frequency to effect cleaning of the teeth. Although such ultrasonic brushes are effective at dislodging plaque and other debris from the teeth, they are not entirely satisfactory in that the dislodged material can resettle in the oral cavity.

U.S. Pat. No. 6,217,328 (Oliver) discloses an oral hygiene system for hospital use which comprises a hand-held cleaning instrument attached to a mobile cart by an accessory hose. The cleaning instrument includes a rotary brush and components for irrigating and suctioning the oral cavity. In addition, it is stated that an ultrasonic cleaner may be located on top of the cart for denture cleaning. Although Oliver does not describe the denture cleaner in detail, such devices are known and typically comprise a bath for submerging dentures, which is subjected to ultrasonic energy to loosen food debris and dirt from the dentures. Because of the bulk of the unit it is not practical for home use, and the use of it requires full or partial assistance by the individual who brings the unit to the patient.

### DISCLOSURE OF INVENTION

The present invention seeks to eliminate, or at least mitigate, the disadvantages of such known oral hygiene devices, or at least provide an alternative. To this end, embodiments provide a means for the effective removal of plaque and debris from teeth and the oral cavity in the form of a device that loosens the plaque and/or other material by irrigation and sonication and then evacuates it by a vacuum system.

According to the present invention, there is provided a toothbrush apparatus comprising a toothbrush head having a bristle portion at its one end comprising bristles, and a handle at its other end; fluid supply means for providing pressurized fluid to the bristle portion, said supply means having an inlet connectable to a source of fluid and an outlet formed in the bristle portion; suction means having an inlet associated with the bristle portion for evacuating waste from the oral cavity of a user for disposal; an ultrasonic transducer for imparting ultrasonic vibrations to the bristles and water medium; and power supply means for supplying electrical power to the ultrasonic transducer.

In preferred embodiments of the invention, the suction means comprises discharge tube means connected at one end to the head and at its other end to a pump for creating a partial vacuum, of approximately 8 inches of mercury, in said discharge tube means so that, when in use, waste can be suctioned into the discharge tube means from the mouth of a user. Preferably, the pump is a fluid-operated venturi-effect pump for receiving liquid from said fluid supply means to create a sufficient vacuum required to remove the liquid with its suspended particulate from the mouth.

The power supply means may comprise a power converter unit having electrical input leads connectable at their distal ends to an external power supply and electrical output leads connected to the transducer, the power converter converting electrical power from said power supply to a suitable high-frequency alternating voltage for powering the ultrasonic transducer.

Both the pump and the transformer may be housed in a supply/storage unit, hereinafter termed simply a “supply unit”. Preferably, the head is detachable from the toothbrush unit and the supply unit includes a storage holder for storing the detachable head, and other similar heads, when not in use. The storage holder may also hold a partial or full denture which can be quickly sterilized before the toothbrush heads are returned to sterilization and storage.

The supply unit may further comprise an ultraviolet light source for irradiating the detachable head when housed in the storage holder. The brush heads and their holding platform may be briefly removed to make room for a quick sterilization of one partial denture and a full denture or one orthodontic appliance.

By reason of the fact that water at normal domestic supply pressure is used, and that a simple venturi pump supplies the suction, the supply unit can be quite compact and light, and does not require its own cart, as in some prior known apparatus.

The bristles are preferably angulated from their attached position on the base of the bristle portion so as to be splayed with their tips diverging outwards. Preferably the bristles are surrounded by a flexible skirt which encloses a major portion of the length of the bristles. The bristles and skirt may be configured so that, as the bristles become splayed apart in use, the edge of the skirt contacts the teeth and/or gums to confine the fluid and enhance the vacuum effect, while not sealing fully.

The invention will become more apparent from the following detailed description, in conjunction with the accompanying drawings which are diagrammatic and not necessarily to scale, of a preferred embodiment of the invention which is described by way of example only.

### BRIEF DESCRIPTION OF THE DRAWINGS

FIG. 1 is a general illustration of a toothbrush apparatus comprising a toothbrush head detachably connected by an umbilical cord to a supply unit;

FIG. 2 is a more detailed, perspective illustration of the toothbrush head and associated parts;

FIG. 2A is a more detailed side illustration of the toothbrush head showing the relationship of the bristles to the skirt;

FIG. 3 is an enlarged perspective illustration of the bristle portion of the toothbrush head;

FIG. 4 is a detail illustration of the bristles and skirt in relation to a molar tooth;

FIG. 5A is a detail illustration of the bristles in relation to the concave side of a tooth and the adjacent gum;

FIG. 5B is a magnified illustration of a portion of FIG. 5A;

FIG. 6A is a schematic representation of the supply unit;

FIG. 6B is one end illustration of the toothbrush head storage unit; and

FIG. 6C is another end illustration of the toothbrush head storage unit.

### BEST MODE(S) FOR CARRYING OUT THE INVENTION

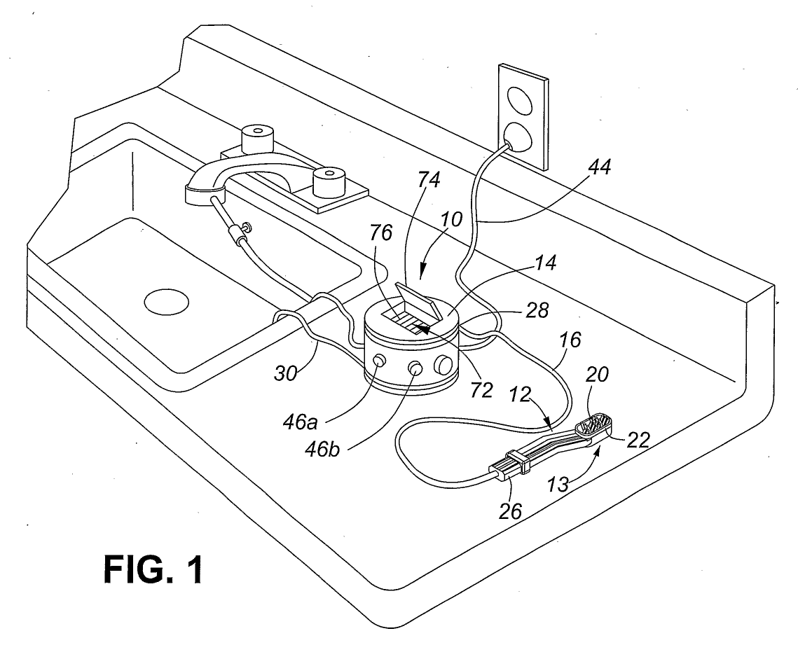

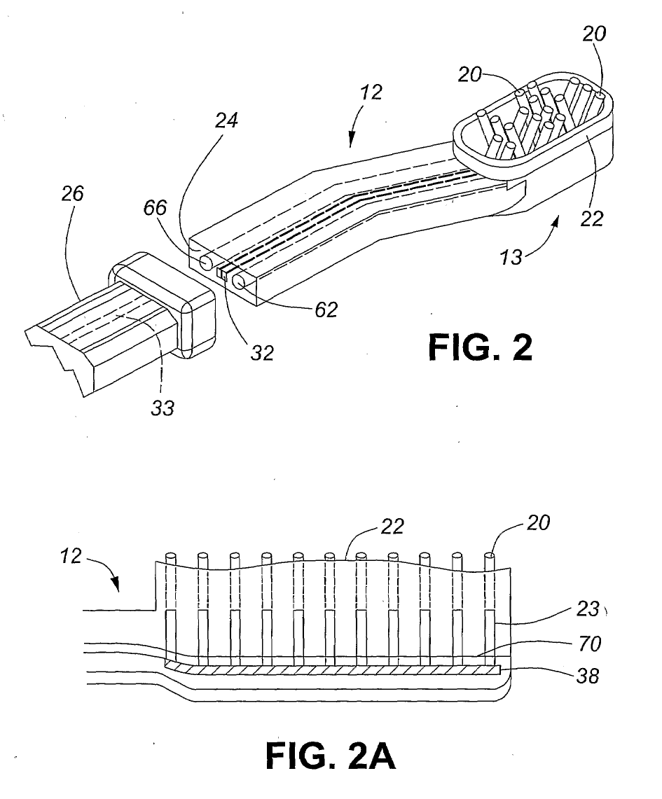

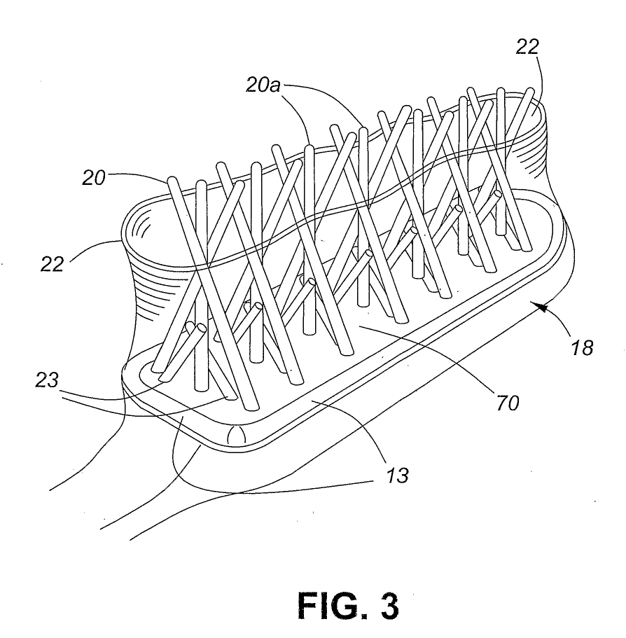

Referring to FIG. 1, a toothbrush apparatus 10 comprises a powered toothbrush head 12 detachably connected to a supply unit 14 by an umbilical cord 16. A bristle portion 13 of head 12 has a compartment 18 comprising bristles 20 projecting from a pad 70 and surrounded by a skirt 22, these parts being shown in detail in FIGS. 2, 2A and 3. The skirt 22 is made of flexible material such as rubber-like polyurethane material of appropriate flexibility such as will conform to hard and soft tissues of teeth and gums and yet not be distorted substantially by pressure forces caused by suction, as will be described. The surface of the skirt is minimally absorbent so that sanitation can be readily achieved by the UV sanitation system (to be described) between brushings. As seen in FIG. 2A, the skirt 22 has a length (height above the pad 70) such as to enclose the major length of the bristles, preferably at least 75%, but more preferably at least 85% of that length, but is slightly shorter than the bristles when they are at rest so that at maximum deflection the skirt edge and bristle tips are at the same level.

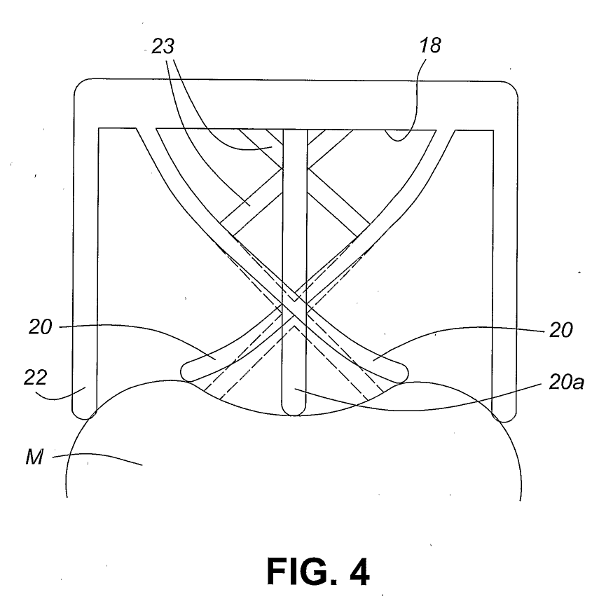

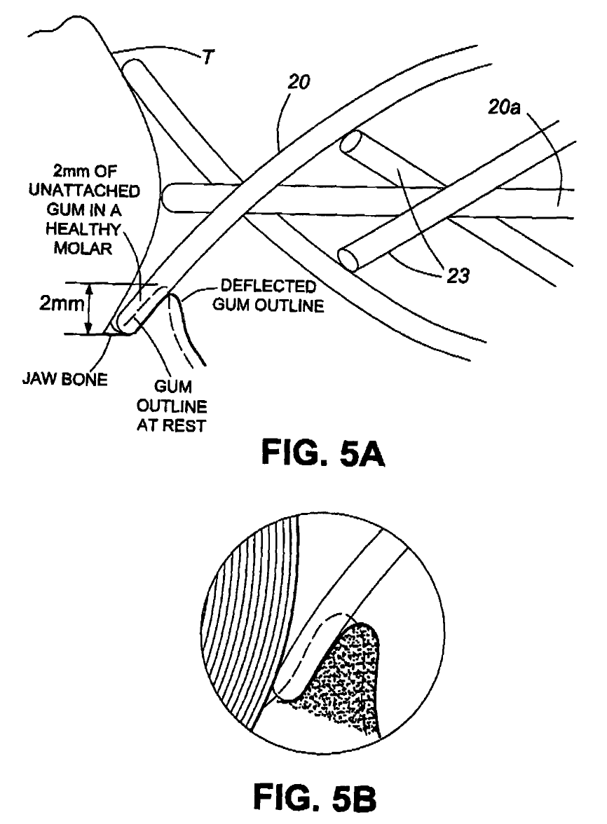

As also shown in FIGS. 2 and 3, the bristles 20 are splayed or angled sideways. Specifically, alternate rows of bristles are angled towards opposite sides of the bristle portion 13, while projecting perpendicularly to the pad 70 when seen in side view as in FIG. 2A. FIG. 3 is simplified to avoid clutter; in fact there is an additional row of seven central bristles 20a (seen in FIGS. 4 and 5A) running from end to end, these bristles 20a projecting perpendicularly from the base of the head. The bristle tips have an undulating concave-convex contour determined by average cheek side convexities of two adult bicuspids and the two first and second deciduous molars for the child's brush.

Another feature is the supply of water, or other cleansing liquid, via a plurality of water tubes 23 in the bristle portion and which are associated with the bristles, and which are supplied by water supply tubing 62 in the head 12. The water tubes 23 project outwards from the base of the bristle portion 13, one water tube preferably being provided for each angled bristle. The tubes 23 are about one half the length of the associated angled bristles, and are themselves angled at about 45 degrees to the base, the tubes and associated bristles being oppositely angled so that a tube projecting from one side of the base ends near to a central region of a bristle projecting from the other side of the base, and is roughly perpendicular to that bristle. The tube outlet ends are close to the centers of the associated bristles so as almost to contact the bristles during maximum deflection, the tubes thus directing the incoming water onto the bristles and onto the hard and soft tissues of the mouth. More details of the arrangement are described below with reference to FIGS. 4 and 5A.

The base of the bristle portion 13 is in the form of a hollow sump which collects water which has been sprayed by tubes 23 and which is connected to a suction waste tube, 66 in the head 12. At the base of the sump is an ultrasonic transducer 38, typically a piezoelectric transducer, that underlies the pad 70 from which project the bristles 20, so that the pad can impart high frequency vibrations to the bristles and to water within the bristle portion 13. The transducer causes longitudinal vibrations in the bristles. Depending upon the nature of the transducer 38, it may be provided with a low voltage signal supply modulated at the required ultrasonic frequency, or may be provided with a D.C. voltage with the transducer 38 converting it to an ultrasonic frequency. The transducer 38 receives current via electrical leads 32 extending along the head 12 and which connect via contacts to leads 33 extending along the umbilical cord 16, in turn connected to the power supply 34 in the supply unit 14 (described below). Electricity is supplied to the power supply unit 34 by input leads 42 in a power cord 44, these leads 42 being connectable to a conventional power source via a plug. A switch 46a is mounted on the front of supply/storage unit 14 for controlling the supply of current to the transducer 38. A second switch 46b controls the degree of vacuum.

The inner end 24 of the head 12 is a snap fit into a complementary connector part 26 at the outer end of the umbilical cord 16. The inner end of the umbilical cord 16 is connected to a socket 28 on the supply unit 14. This unit 14 provides electrical power and irrigation fluid, specifically water, to the toothbrush head 12 via the umbilical cord 16. The umbilical cord 16 also extracts waste from the head 12 via the tube 66, and discharges it, typically into a sink, through a drain tube 30. Thus, the umbilical cord 16 includes tubes 62 and 66 which respectively supply water to the head 12 and remove waste, and also provides electrical leads 33 to supply high-frequency alternating electrical power to the head 12 from the supply unit 14.

FIGS. 4, 5A and 5B show further details of the brush head bristles 20 and water supply tubes 23, in relation to teeth. As shown in FIG. 4, the toothbrush head is applied to the biting surface of a molar M, with the outer edges of skirt 22 in contact, or near contact, with the tooth, the slanted bristles 20 contacting the tooth while being deflected from the “at rest” condition which is shown in broken lines. The central bristles 20a are little deflected. The irrigation tubes 23 are slanted in this view so that they supply water to mid portions of the slanted bristles 20. A bicuspid tooth would be similar but narrower.

FIG. 5A shows the action of the bristles on the cheek side of a tooth T; here the skirt is not shown for simplicity. The slanted bristles 20 are deflected as before, and the central bristles 20a are not significantly deflected. As will be seen in the lower part of the drawing, and in the magnified view in FIG. 5B, some of the bristles will enter gum pockets at the sides of the teeth. It is within these 2 mm natural pockets that gum disease starts when plaque is not removed. Even with diseased gums the bristles can still reach the bases of the pockets. Similar results will be achieved on the tongue side of the teeth, which have the same average convexity except at the back of the front teeth which are concave; the bristles would still be effective with this concavity.

The bristles 20 within the skirt 22 are splayed with their tips projecting outwards so that when in use, the bristle portion 13 is pressed gently against the teeth and adjacent gum. This has two benefits. The first is that it will allow the skirt 22 to close onto the teeth and gum to ensure that the vacuum discharge works efficiently. It should be appreciated that the skirt should not seal onto the teeth and gum because the seal could cause the exhaust flow to cease. Rather, the object is to restrict the opening between the skirt and teeth/gum so that the evacuation force, and hence flow, is adequate. The second benefit is that the tips of the splayed bristles are oriented at a better angle to remove plaque or food residue at the joint between the teeth and the gum, as shown in FIG. 5.

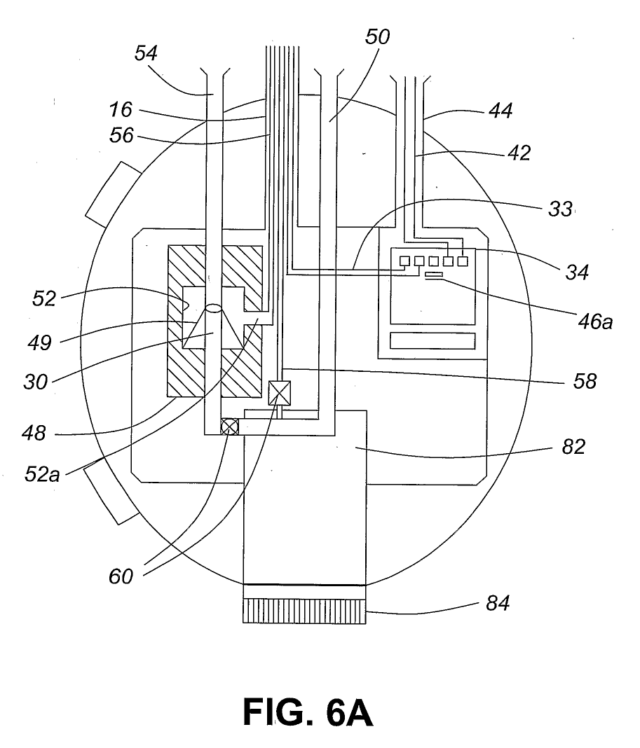

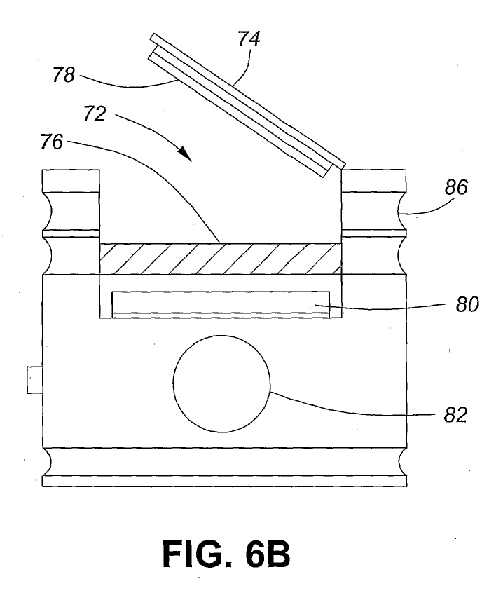

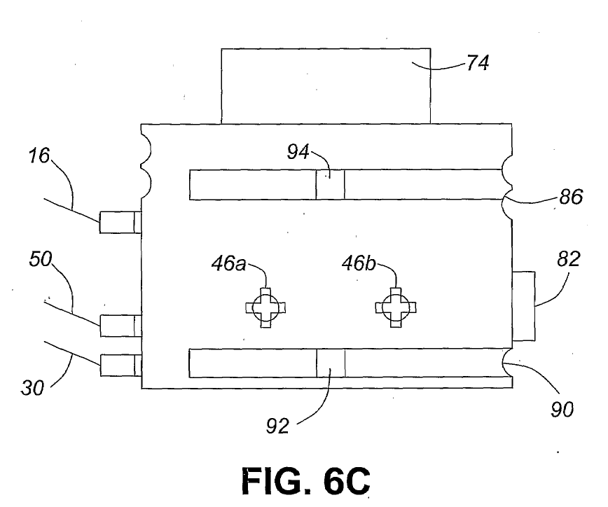

FIGS. 6A, 6B, and 6C show details of the supply unit 14.

As shown in detail in FIG. 6A, the lower portion of the unit comprises a power supply converter unit 34 including a transformer and conversion circuitry for converting the usual household power supply of, say, 110 volts, to an ultrasonic frequency low voltage, e.g., about 1.6 volts, 20,000 Hz, to power the ultrasonic transducer 38 disposed in the head 12.

As also illustrated in FIG. 6A, a fluid-driven, venturi-effect vacuum pump 48 is coupled to a main fluid supply line 50 which, in use, is connected to a supply of water under pressure, conveniently a domestic water supply as shown in FIG. 1. The venturi pump 48 has a nozzle 49 surrounded by a compartment 52, the nozzle being directed towards a central outlet pipe 54 connected to the drain tube 30. The action of nozzle 49, when water is supplied thereto, causes negative pressure in the compartment 52, which in turn creates a partial vacuum in a discharge tube 56 connected to side port 52a of the compartment 52. The contaminated water from the head 12 is drawn via the discharge tube 56 into the compartment 52 and discharged through outlet pipe 54 along with water from the main supply line 50. A branch supply line 58 communicating with the supply line 50 provides irrigation water to the bristle portion 13 via the water supply line 62. Water flow regulators 60 are provided in the branch supply line 58 and can be used to adjust the water pressure supplied to the head 12, thus compensating for different water pressures.

The branch supply line 58 extends along the umbilical cord 16 to the connector 26. Referring to FIG. 2, the connector 26 couples the branch supply line 58 to the supply tube 62 in: the head 12. The discharge tube 56 couples to the shorter discharge tube 66 in the head 12 which communicates with the pump between the skirt 22 and the plastics pad 70 holding the bristles 20. Since the discharge tube 56 is connected to the venturi pump 48 which creates a negative pressure, irrigation water, together with debris, such as plaque, tartar, saliva and so on is evacuated via tubes 66 and 56 and discharged via drain tube 30.

As shown in FIGS. 1, 6B and 6C, a storage holder 72 is provided at the top of the supply/storage unit 14 to house/store up to four interchangeable heads and, optionally, other dental implements. When the lid 74 of the unit 14 is opened, a rack 76 in the holder moves toward the top of the unit with the lid, thereby making it easier for a user to remove a suitable head or dental implement. This storage holder 72 also is provided with a UV light source 78 mounted under the lid 74 to sterilize the upper surfaces of the heads and/or other contents while stored in the compartment under the lid. The UV light source 78 becomes illuminated when the lid of the supply unit is closed, and remains on while the unit is being used. A further UV light source 80 is mounted at the base of the storage chamber, this turns on and remains on while the unit it being used to sterilize the lower surfaces of the heads, etc. It may also irradiate water coming into the unit and sterilize waste leaving the unit, by having transparent sections of the pipes passing under the source 80. It is preferable to sterilize the waste water to make this safe, in case a child should suck on the outlet line.

Although the above-described embodiment makes use of a drain tube 30 to discharge waste liquid, a portable unit could have a discharge tank for temporary storage. Similarly, a reservoir and pump can be provided for water supply, and temperature control for the water supply can be provided if desired. The water supply can be continuous or it can be pulsed or intermittent as in the background art as discussed above. Similarly, the suction can be continuous or can be controlled to be intermittent; for example the water supply and suction could occur alternately in a repetitive cycle.

In order to remove chlorine and other contaminants from water entering the unit, the water supply line 50 may be connected to a conventional charcoal filter 82, shown in FIG. 6A, which, in turn, is attached to a water faucet. FIGS. 6B and 6C show a suitable position for the charcoal filter lid 84. Although the embodiment described above employs LTV light to sterilize the heads, the supply/storage unit 14 can be used for cleaning and sanitizing dentures and other devices worn in the mouth.

Optionally, the drain tube 30 may be secured to the toothbrush unit by a spiral groove 90 and clip 92 or other securing means. The power cord 44 may be secured to the unit in a similar manner. This allows for compact storage of the toothbrush unit when not in use. The sides of the unit are provided with concavities 86 and a clip 94 for storing the umbilical cord 16.

It should be appreciated that various electrical to mechanical transducers may also be utilized instead of piezoelectric transducers, such as piezo- or magneto-strictive devices.

### INDUSTRIAL APPLICABILITY

Advantageously, embodiments of this invention provide a toothbrush unit for home use that loosens and suspends plaque by ultrasonic action and irrigation and then removes it by suction. This results in the removal of a large amount of plaque since it is effectively dislodged from the surfaces of the teeth and crevices thereby making it available for suction removal. Also, by suctioning the loosened plaque, it is prevented from being retained in the mouth and allowed to resettle in crevices between the teeth. Moreover, embodiments of this invention provide a toothbrush unit that is of simple construction and easy to operate in relation to known sonic toothbrushes.

Particular advantages and features of an embodiment of the invention include the following:

### 1. No Messy, Expensive Toothpaste Needed or Allowed:

Toothpaste could inhibit the efficient removal of plaque by the vacuum system. The chemicals in toothpaste that inhibit plaque retention and harden enamel can be just as effectively administered by an appropriate mouth rinse following brushing

### 2. No More Flossing:

Flossing loosens plaque and for the most part forces it into the unattached glm collar around the tooth and, for those with diseased gum pockets, pushes the plaque deep into these pockets where it is very difficult to remove. It is almost impossible to effectively floss without precipitating some bleeding especially, in those mouths that have some degree of gingivitis (inflammation of the gums). This can lead to serious consequences for patients with certain heart defects and for those who have had artificial joint and hip replacements within the previous 2 years. Harmful bacteria living in plaque are introduced into the bloodstream if any bleeding occurs during flossing or even brushing too vigorously.

### 3. No Rinsing:

The plaque is suspended in the sterile water coming from the toothbrush and removed to waste by the vacuum system in a manner similar to that used by a hygienist.

### 4. No Chlorine in the Water:

The carbon filter removes chlorine from the water entering the toothbrush power unit.

### 5. Sterile Toothbrush Each Time and Sterile Water Each Time:

The UV lights in the four toothbrush, partial or full denture, or orthodontic appliances storage box in the power unit automatically sterilize the brush heads, etc. after each use and, as well, the outgoing waste water, so that no biofilm has a chance to grow harmful bacteria within the unit.

Embodiments of this invention also provide a toothbrush unit that can be placed in close proximity to the gums so that maximum plaque removal is achieved Furthermore, in embodiments of this invention, there is provided a toothbrush unit that is powerful enough to clean tooth enamel without injuring the surrounding tissues since the user only needs to position and hold the toothbrush against the surface of the teeth. This is in contrast to conventional toothbrushes which can cause damage to the gum since they require the user to manually scrub the teeth. Moreover, some powered toothbrushes may be misused easily and cause gum damage, such as receding gums. In addition, embodiments of the invention provide for a toothbrush unit that is sanitary by utilizing a UV light source that sterilizes toothbrush heads and, optionally, incoming water and outgoing waste.

Accordingly, aspects of this invention include the following:

1. A toothbrush apparatus comprising: a toothbrush head (12) having at one end a bristle portion (13) with bristles (20) projecting from a base, and having a handle at its other end; fluid supply means (50,58,62) for providing pressurized fluid to the bristle portion, said supply means (50,59,62) having an inlet connectable to a source of fluid and an outlet formed in the bristle portion; a flexible skirt (22) surrounding said bristles and forming a sump at the base of the bristles for reception of liquid; suction means (48,56,66) having an inlet associated with the base of the bristle portion (13) for evacuating waste from the oral cavity of a user for disposal; an ultrasonic transducer (38) for imparting ultrasonic vibrations to the bristle portion; and power supply means (32,34,36,42,44) for supplying electrical power to the ultrasonic transducer (38).
2. A toothbrush apparatus according to paragraph 1, wherein said flexible skirt (22) encloses a major portion of the height of the bristles above said base.
3. A toothbrush apparatus according to paragraph 1 or paragraph 2, wherein said bristles (20) are splayed to one or other of the sides of the toothbrush head.
4. A toothbrush apparatus comprising: a toothbrush head (12) having at one end a bristle portion (13) with bristles (20) projecting from a base, and having a handle at its other end; fluid supply means (50,58,62) for providing pressurized fluid to the bristle portion, said supply means (50,58,62) having an inlet connectable to a source of fluid and an outlet formed in the bristle portion; suction means (48,56,66) having an inlet associated with the base of the bristle portion (13) for evacuating waste from the oral cavity of a user for disposal; an ultrasonic transducer (38) for imparting ultrasonic vibrations to the bristle portion along the longitudinal axes of the bristles; and power supply means (32,34,36,42,44) for supplying electrical power to the ultrasonic transducer (38); and wherein said bristles (20) are splayed to one or other of the sides of the toothbrush head.
5. A toothbrush apparatus according to paragraph 4, wherein said bristles include a series of rows of splayed bristles which are alternately angled to opposite sides of the toothbrush head, and a longitudinal central row of bristles which are substantially perpendicular to the base.
6. A toothbrush apparatus according to paragraph 1 or paragraph 4, wherein said outlet for the fluid supply means is provided by a plurality of outlet tubes in said bristle portion.
7. A toothbrush apparatus comprising: a toothbrush head (12) having at one end a bristle portion (13) with bristles (20) projecting from a base, and having a handle at its other end; fluid supply means (50,58,62) for providing pressurized fluid to the bristle portion, said supply means (50,58,62) having an inlet connectable to a source of fluid and an outlet formed in the bristle portion; suction means (48,56,66) having an inlet associated with the base of the bristle portion (20) for evacuating waste from the oral cavity of a user for disposal; an ultrasonic transducer (38) for imparting ultrasonic vibrations to the bristle portion along the longitudinal axes of the bristles; and power supply means (32,34,36,42,44) for supplying electrical power to the ultrasonic transducer (38); and wherein said outlet for the fluid supply means is provided by a plurality of outlet tubes (23) in said bristle portion.
8. A toothbrush apparatus according to paragraph 7, wherein said bristles include bristles (20) which are angled to one or other side of said toothbrush head, and wherein one of said outlet tubes (23) is associated with each of the angled bristles.
9. A toothbrush apparatus according to paragraph 8, wherein said outlet tubes (23) are approximately one half the length of the associated bristles.
10. A toothbrush apparatus according to any of paragraphs 1, 4 or 7, wherein said suction means is a venturi pump (48).
11. A toothbrush apparatus according to paragraph 10, wherein the fluid supply means (50,58,62) comprises a main fluid supply line (50) that is connectable at its one end to a faucet and at its other end to said venturi pump (48); and a branch supply line (58) communicating with the main fluid supply line (50), said branch supply line (58) providing fluid to the head (12).
12. A toothbrush apparatus according to paragraph 11, wherein the branch supply line (58) is connectable at its distal end portion to a second fluid supply line (62) disposed in the head (12) for providing fluid to said bristle portion (13).
13. A toothbrush apparatus according to any one of paragraphs 1, 4, 7 or 11, wherein the power supply means comprises a power converter unit (34) having electrical input leads (42) connectable at their distal ends to an external power supply and electrical output leads (42) connected to the transducer (38), the power converter (34) converting electrical power from said power supply to a suitable high-frequency alternating voltage for powering the ultrasonic transducer.
14. A toothbrush apparatus according to paragraph 13, wherein both the pump (48) and the power converter unit (34) are housed in a supply unit (14), and wherein said head (12) is detachable from the remainder of the toothbrush apparatus, and wherein the supply unit (14) comprises a storage holder (72) for storing said detachable head (12) when not in use.
15. A toothbrush apparatus according to paragraph 14, wherein the supply unit (14) further comprises an ultraviolet light source for irradiating said detachable head (13) when stored in said storage holder (72).
16. A toothbrush apparatus according to any one of paragraphs 1 to 15, wherein the transducer (38) is a piezoelectric crystal disposed in the base beneath the bristles (20).
17. A toothbrush apparatus comprising: a toothbrush head (12) having at one end a bristle portion (13) with bristles (20) projecting from a base, and having a handle at its other end; fluid supply means (50,58,62) for providing pressurized fluid to the bristle portion, said supply means (50,58,62) having an inlet connectable to a source of fluid and an outlet formed in the bristle portion; suction means (48,56,66) having an inlet associated with the base of the bristle portion (13) for evacuating waste from the oral cavity of a user for disposal; an ultrasonic transducer (38) for imparting ultrasonic vibrations to the bristle portion; and power supply means (32,34,36,42,44) for supplying electrical power to the ultrasonic transducer (38).

Although an embodiment of the invention has been described and illustrated in detail, it is to be clearly understood that the same is by way of illustration and example only and not to be taken by way of the limitation, the spirit and scope of the present invention being limited only by the appended claims.

1. A toothbrush head comprising:
a bristle portion having bristles projecting from a base;
a handle extending from the bristle portion;
fluid supply means for supplying fluid from an inlet to the bristle portion;
a skirt surrounding the bristles and forming a swnp at their base;
fluid removal means for removing fluid from the sump to an outlet;
an ultrasonic transducer for imparting ultrasonic vibrations to the bristle portion; and
means for supplying electrical power to the ultrasonic transducer.

2. A toothbrush head as claimed in claim 1, wherein the skirt comprises a flexible material.

3. A toothbrush head as claimed in claim 1, wherein the skirt surrounds the bristles for at least about 75% of their height above the base.

4. A toothbrush head as claimed in claim 1, wherein the skirt surrounds the bristles for at least about 85% of their height above the base.

5. A toothbrush head as claimed in claim 1, wherein the bristles include bristles extending at angles towards opposite sides of the bristle portion.

6. A toothbrush head as claimed in claim 5, wherein the bristles also include bristles projecting perpendicular to the base.

7. A toothbrush head as claimed in claim 1, wherein the bristles comprise two rows of bristles extending along a length of the bristle portion, with bristles of said two rows extending from one side of the bristle portion at the base towards an opposite side of the bristle portion at an opposite end of the bristles.

8. A toothbrush head as claimed in claim 7, wherein the bristles comprise at least one further row of bristles extending along a length of the bristle portion between said two rows of bristles, bristles of said at least one further row of bristles projecting substantially perpendicularly from said base.

9. A toothbrush head as claimed in claim 1, wherein the fluid supply means comprises a plurality of tubes for supplying fluid to points adjacent to and intermediate the ends of the bristles.

10. A toothbrush head as claimed in claim 7, wherein the fluid supply means comprises a respective tube for supplying fluid to a point adjacent to and intermediate the ends of each of the bristles of said two rows of bristles.

11. A toothbrush apparatus comprising a toothbrush head and a supply unit, the toothbrush head comprising
a bristle portion having bristles projecting from a base;
a handle extending from the bristle portion;
fluid supply means for supplying fluid from an inlet to the bristle portion;
a skirt surrounding the bristles and forming a sump at their base;
fluid removal means for removing fluid from the sump to an outlet;
an ultrasonic transducer for imparting ultrasonic vibrations to the bristle portion; and
means for supplying electrical power to the ultrasonic transducer,
the supply unit comprising:
a cord including a fluid supply tube for coupling to said inlet of the toothbrush head, a fluid removal tube for coupling to said outlet of the toothbrush head, and electrical leads for supplying electrical power for the ultrasonic transducer of the toothbrush head;
means for supplying fluid to the fluid supply tube;
means for applying suction to the fluid removal tube; and
means for supplying electrical power for the ultrasonic transducer to the electrical leads.

12. A toothbrush apparatus as claimed in claim 11, wherein the means for applying suction to the fluid removal tube comprises a venturi pump.

13. A toothbrush apparatus as claimed in claim 12, wherein the means for supplying fluid to the fluid supply tube is also arranged to supply fluid to the venturi pump.

14. A toothbrush apparatus as claimed in claim 11, wherein the means for supplying electrical power comprises a power converter for converting electrical power from an external source to an alternating voltage for powering the ultrasonic transducer.

15. A toothbrush apparatus as claimed in claim 11, wherein the toothbrush head is detachable from the cord and the supply unit includes a holder for storing at least one toothbrush head.

16. A toothbrush apparatus as claimed in claim 15, wherein the supply unit includes an ultraviolet light source for irradiating a toothbrush head stored in said holder.

17. A toothbrush apparatus as claimed in claim 11, wherein the supply unit comprises an ultraviolet light source for irradiating fluid supplied to the fluid supply tube and/or fluid from the fluid removal tube.
